(19) United States
(12) Patent Application Publication
(10) Pub. No.: US 2014/0161813 A1
(43) Pub. Date: Jun. 12, 2014

(54) METHODS FOR THE DIAGNOSIS, TREATMENT AND MONITORING OF CANCER

(71) Applicant: Bauer Research Foundation, Akron, OH (US)

(72) Inventors: Joseph A. BAUER, Port St. Lucie, FL (US); Annette M. SYSEL, Vero Beach, FL (US)

(73) Assignee: Bauer Research Foundation, Akron, OH (US)

(21) Appl. No.: 14/103,680

(22) Filed: Dec. 11, 2013

Related U.S. Application Data
(60) Provisional application No. 61/849,376, filed on Jan. 25, 2013.
(60) Provisional application No. 61/736,415, filed on Dec. 12, 2012.

(51) Int. Cl. A61K 31/7056; G01N 33/574; C12Q 1/68; A61K 45/06

(52) U.S. Cl. CPC A61K 31/7056; A61K 45/06; G01N 33/57484; C12Q 1/68
USPC 424/155.1; 514/52; 435/7.23; 506/9; 435/7.92; 435/6.12

(57) ABSTRACT

New methods for diagnosing, treating, and monitoring cancer have been developed based on the expression level of markers, such as transcobalamin II (TCII), transcobalamin II receptor (TCIIR), Ki-67, megalin, cubilin, amnionless and/or asialoglycoprotein receptors. For example, a cancer diagnosis can be made by determining the level of TCII, TCIIR, Ki-67, megalin, cubilin, amnionless and/or asialoglycoprotein receptor expression in a test sample from the subject and in a reference sample, and diagnosing the subject as having cancer if the level of expression of transcobalamin II (TCII), transcobalamin II receptor (TCIIR), Ki-67, megalin, cubilin, amnionless, an asialoglycoprotein receptor, or a combination thereof, is statistically significantly different than the respective reference sample.

### CROSS-REFERENCE TO RELATED APPLICATIONS

This application claims the benefit of U.S. Provisional Application Ser. No. 61/736,415, filed Dec. 12, 2012, and U.S. Provisional Application Ser. No. 61/849,376, filed Jan. 25, 2013, both of which are incorporated herein by reference.

### FIELD OF THE INVENTION

The present invention relates to methods for diagnosing, treating, and monitoring cancer based on the expression level of marker proteins that bind to vitamin B12, such as transcobalamin II (TCII), transcobalamin II receptor (TCIIR), megalin, cubilin, amnionless and/or asialoglycoprotein receptors, and/or a proliferation marker such as Ki-67.

### BACKGROUND

Despite numerous advances in medical research, cancer remains one of the leading causes of death in the United States. Therefore, the use of biologic markers to more accurately diagnose and predict effective therapy is receiving increased interest in both human and veterinary cancer medicine. The ability to direct an individualized diagnostic and therapeutic plan can increase the probability of treatment success, maximize use of valuable treatment time and minimize financial costs associated with ineffective diagnostics or drugs. For example, Human Epidermal Growth Factor Receptor 2 (HER-2), a protein encoded by the ERBB2 gene, has become an important biomarker and target of therapy in human breast cancer patients. In veterinary cancer medicine, mutation of the proto-oncogene c-Kit is one of the few biologic markers currently used clinically, and is useful in determining the likelihood of tumor response to tyrosine kinase inhibitors such as Palladia®. Development of new, accurate markers is critical to increasing our ability to appropriately select diagnostic and therapeutic modalities for individual patients, as well as to monitor their course of disease. The ability to do so will ultimately improve their quality of life.

Vitamin B12 (cobalamin) plays an important role in DNA synthesis and cell division. In particular, vitamin B12 plays an important role in the differentiation, proliferation and metabolic stability of cells. Once absorbed into the bloodstream, vitamin B12 is bound to transcobalamin II (TCII), a non-glycosylated transport protein found in the blood. The vitamin B12-TCII complex is then transported to cells and undergoes receptor-mediated endocytosis into cells via the transcobalamin II receptor (TCIIR), a specific vitamin B12 receptor on the cells' surface. Thus, the TCII and TCIIR delivery system plays a significant role in vitamin B12 uptake by cells. Other receptors that take up vitamin B12 include megalin, cubilin, amnionless and asialoglylcoprotein receptors.

While all living cells require vitamin B12 for survival, rapidly dividing cancer cells require especially high levels of vitamin B12 to sustain their rapid growth. Studies have demonstrated that vitamin B12 is taken up in greater quantities by tumor cells relative to normal tissues (Oleson et al., “Effect of pteroylglutamic acid and vitamin B12 on growth of Rous tumor implants,” Proc Soc Exp Biol Med, 1949, 71(2): p. 226; Cooperman et al., “Distribution of radioactive and nonradioactive vitamin B12 in the dog,” J Biol Chem, 1960, 235: p. 191-4; Flodh et al., “Accumulation of labelled vitamin B12 in some transplanted tumours,” Int J Cancer, 1968, 3(5): p. 694-9; Ramanujam et al., “Expression of cobalamin transport proteins and cobalamin transcytosis by colon adenocarcinoma cells,” Am J Physiol, 1991, 260 (3 Pt 1): p. G416-22).

Vitamin B12 bound to various molecular imaging agents has been used to detect tumors in mice, rats, and humans (Collins et al, “Transcobalamin II receptor imaging via radiolabeled diethylene-triaminepentaacetate cobalamin analogs,” J Nucl Med, 1997, 38(5): p. 717-23; Collins et al., “Tumor imaging via indium 111-labeled DTPA-adenosylcobalamin,” Mayo Clin Proc, 1999, 74(7): p. 687-91; Collins et al., “Biodistribution of radiolabeled adenosylcobalamin in patients diagnosed with various malignancies,” Mayo Clin Proc, 2000, 75(6): p. 568-80). Fluorescent-labeled vitamin B12 has been used as a minimally invasive method to outline lymph node drainage patterns (McGreevy et al., “Minimally invasive lymphatic mapping using fluorescently labeled vitamin B12,” J Surg Res, 2003, 111(1): p. 38-44). Indium-111 labeled vitamin B12 has been used to image primary tumors as well as lung metastases in a dog (Whittemore et al., “Indium-111 labeled vitamin B12 imaging of a ciliary adenoma with concurrent grade 2 soft tissue sarcoma of the leg in a Labrador Retriever,” Vet Ophthalmol, 2004, 7(3): p. 209-12). In human patients, the biodistribution of radiolabeled vitamin B12 has been evaluated in primary and recurrent tumors of the breast, lung, colon, skin, thyroid and central nervous system (Collins et al, “Biodistribution of radiolabelled adenosylcobalamin in patients diagnosed with various malignancies, “Mayo Clin Proc 2000; 75 (6): p. 568-580). The use of radiolabeled vitamin B12 is receiving increased attention for use in imaging for detection of primary and metastatic tumors, for delineation of lymph node drainage and for definition of margins between normal and cancerous tissues during surgical resection.

Additionally, novel vitamin B12-based compounds are being developed as anti-cancer agents. For example, nitrosylcobalamin (NO-Cbl) is a newly developed anti-cancer compound that consists of vitamin B12 bound to nitric oxide (Bauer, “Synthesis, characterization and nitric oxide release profile of nitrosylcobalamin: a potential chemotherapeutic agent,” Anticancer Drugs, 1998, 9(3): p. 239-44). The addition of nitric oxide to the cobalamin molecule represents a very subtle change to the chemical structure of cobalamin, and thus cells cannot distinguish between NO-Cbl and vitamin B12. Once NO-Cbl is internalized within cancer cells, nitric oxide is liberated from vitamin B12, and its release inhibits cancer cell metabolism and leads to cellular apoptosis and necrosis (Gross et al., “Nitric oxide: pathophysiological mechanisms,” Ann Rev Physiol, 1995, 57: p. 737-69; Burney et al., “The chemistry of DNA damage from nitric oxide and peroxynitrite,” Mutat Res, 1999, 424 (1-2): p. 37-49; Chawla-Sarkar et al., “Suppression of NF-kappa B survival signaling by nitrosylcobalamin sensitizes neoplasms to the anti-tumor effects of Apo2L/TRAIL,” J Biol Chem, 2003, 278(41): p. 39461-9; Tang et al., “Nitrosylcobalamin promotes cell death via S nitrosylation of Apo2L/TRAIL receptor DR4,” Mol Cell Biol, 2006, 26(15): p. 5588-94). Intracellular nitric oxide release results in minimal systemic toxicity and minimal effects on the normal cell population. Thus, a major advantage of NO-Cbl is its tumor-specific accumulation. The National Cancer Institute Developmental Therapeutics Program independently tested NO-Cbl in a human tumor 60 cell line screen and in general, colon, ovarian and breast carcinomas as well as central nervous system tumors were most responsive to the antigrowth effects of NO-Cbl (Bauer et al., “Effects of interferon beta on transcobalamin II-receptor expression and antitumor activity of nitrosylcobalamin,” J Natl Cancer Inst, 2002, 94(13): p. 1010-9). More recently, a case study in dogs showed impressive anti-tumor efficacy of NO-Cbl in the treatment of thyroid carcinoma, malignant peripheral nerve sheath tumor, anal gland adenocarcinoma and spinal meningioma with no evidence of systemic or local toxicity (Bauer et al., “Anti-tumor effects of nitrosylcobalamin against spontaneous tumors in dogs,” Invest New Drugs, 2010, 28(5): p. 694-702). On extended follow-up of treated dogs, daily NO-Cbl treatment ranging in duration from 6 to 15 months was able to completely eliminate the tumor in 3 of 4 dogs; in the remaining 1 dog, daily treatment with NO-Cbl resulted in a 77% decrease in tumor size over the course of 6 months, but the dog was humanely euthanized at that point due to progression of severe arthritis that had been diagnosed prior to initiation of NO-Cbl therapy and that had become unresponsive to standard arthritis treatments. NO-Cbl treatment was able to increase the lifespan of each dog and to improve their quality of life without any of the adverse side effects typically associated with current chemotherapy or radiation regimes. The above references are hereby incorporated by reference in their entirety.

Additionally, U.S. Pat. No. 5,936,082 describes the therapeutic effectiveness of vitamin B12-based compounds. In particular, nitrosylcobalamin (NO-Cbl) was evaluated for its chemotherapeutic effect. U.S. Pat. No. 6,752,986 describes the effect of certain cytokines on the uptake and/or activity of vitamin B12 and vitamin B12 analogs, homologs and derivatives. U.S. Pre-grant Publication No. 2008/0138280 discloses therapeutic compositions comprising cobalamin drug conjugates and methods of treating tumors using the therapeutic compositions. The above patent documents are hereby incorporated by reference in their entirety.

Ki-67 is a protein known to be associated with cellular proliferation. It is present during all active phases of the cell cycle (G₁, S, G₂ and mitosis) but is absent from resting cells (G₀). Ki-67 protein expression is required for progression through the cell-division cycle, making it an excellent marker of the proliferation status of a given cell population. Ki-67 expression has been measured in both human and animal tumors, and has been used to help differentiate benign from malignant tissue, establish tumor grade, identify malignant transformation of benign tumors, aid in selection of treatment protocols, measure treatment response, determine prognosis, and monitor for recurrent or metastatic disease.

Although previous studies have indicated that TCII or TCIIR may be upregulated in cancer cells, heretofore it has not been known whether TCII or TCIIR can be used as a reliable marker for diagnosing cancer, for assisting with selection of a treatment regime(s) or for monitoring the progression of cancer.

### SUMMARY OF THE INVENTION

Preferred embodiments of the invention provide methods for diagnosing and treating cancer in a subject based on the expression levels of transcobalamin II (TCII), transcobalamin II receptor (TCIIR), Ki-67, megalin, cubilin, amnionless and/or an asialoglycoprotein receptor in a test sample. Preferably, the expression levels of at least two of the markers, such as TCII and TCIIR, are evaluated. In other embodiments, three, four, five, six, seven, or more of these markers can be evaluated.

According to one embodiment of the invention, the method for diagnosing cancer in a subject comprises a) determining the level of expression of transcobalamin II (TCII), transcobalamin II receptor (TCIIR), Ki-67, megalin, cubilin, amnionless and/or an asialoglycoprotein receptor in a test sample from the subject; b) determining the level of expression of transcobalamin II (TCII), transcobalamin II receptor (TCIIR), Ki-67, megalin, cubilin, amnionless and/or an asialoglycoprotein receptor in a reference sample; and c) diagnosing the subject as having cancer if the level of the test sample is statistically significantly different than the level of the reference sample.

According to another embodiment of the invention, the method for treating cancer in a subject comprises: a) determining the level of expression of transcobalamin II (TCII), transcobalamin II receptor (TCIIR), Ki-67, megalin, cubilin, amnionless and/or an asialoglycoprotein receptor in a test sample from the subject; b) determining the level of expression of transcobalamin II (TCII), transcobalamin II receptor (TCIIR), Ki-67, megalin, cubilin, amnionless and/or an asialoglycoprotein receptor in a reference sample; and c) administering a chemotherapeutic agent alone or in combination with one or more therapeutic agents to the subject if the level of the test sample is statistically significantly different than the level of the reference sample.

According to another embodiment, the present invention provides a method for monitoring the progression of cancer in a subject. According to one preferred embodiment of the invention, the method for monitoring the progression of cancer in a subject comprises: a) determining the level of expression of transcobalamin II (TCII), transcobalamin II receptor (TCIIR), Ki-67, megalin, cubilin, amnionless and/or an asialoglycoprotein receptor in a test sample obtained from the subject at a first time point; b) determining the level of expression of transcobalamin II (TCII), transcobalamin II receptor (TCIIR), Ki-67, megalin, cubilin, amnionless and/or an asialoglycoprotein receptor in a test sample obtained from the subject at a second time point, wherein the second time point is later than the first time point; and c) evaluating the progression of cancer based on the change between level of the first and the second time points. According to one embodiment, the subject is found to have i) a positive prognosis if the second test level is statistically significantly lower or different than the first test level, or ii) a neutral prognosis if the second test level is not statistically significantly different from the first test level, or iii) a negative prognosis if the second test level is statistically significantly higher or different than the first test level.

The term “subject” or “patient” as used herein includes, but is not limited to, mammals such as bovine, canine, caprine, cervine, equine, feline, human, ovine, porcine and primates. Most preferably, the subject is human, feline, or canine.

The term “statistically significantly higher,” “statistically significantly lower,” and “statistically significantly different” as used herein means the p-value of the difference between the two values is less than 0.05.

According to one embodiment, the present invention provides kits useful for screening, diagnosing, monitoring, or determining a prognosis for a cancer patient. A kit can include at least one of the following: (i) one or more labeled or unlabeled polynucleotide probes capable of hybridizing under reduced stringent, stringent, or highly stringent conditions to transcobalamin II (TCII), transcobalamin II receptor (TCIIR), Ki-67, megalin, cubilin, amnionless and/or an asialoglycoprotein receptor mRNA, or (ii) one or more labeled or unlabeled antibodies capable of specifically binding to transcobalamin II (TCII), transcobalamin II receptor (TCIIR), Ki-67, megalin, cubilin, amnionless and/or asialoglycoprotein receptor proteins. The kit may also include a carrier where the polynucleotide probes or antibodies are immobilized and one or more reagents or detection agents for detecting a reaction between the one or more probes or antibodies and target nucleic acids or polypeptides. The kit may also include software and/or instructions to analyze the data generated by using the kit.

### BRIEF DESCRIPTION OF THE DRAWINGS

Color figures were provided in U.S. Provisional Application Ser. No. 61/849,376, filed Jan. 25, 2013, which is incorporated herein by reference.

FIG. 1 shows the expression of TCIIR (protein antibody=CD320) and TCII (protein antibody=TCN2) in thirty-four human tumor xenograft sections: (1) breast carcinoma MCF7/S, (2) breast carcinoma MDA-MB-231, (3) breast carcinoma ZR-75-1, (4) Burkitt's lymphoma Ramos, (5) cervical C-33A, (6) cervical Ca Ski, (7) cervical HeLa, (8) colon HCT-116, (9) colon HT-29, (10) epidermoid carcinoma A431NS, (11) Ewing's sarcoma CHP-100, (12) GBM SF-767, (13) leukemia MDS/SP1, (14) lymphoma Granta-4, (15) lymphoma Raji, (16) melanoma A375, (17) NSCLC H1975, (18) NSCLC MV-522, (19) NSCLC NCl: H460, (20) ovarian 2780AD, (21) ovarian carcinoma NIH-OVCAR-3, (22) pancreatic AsPC-1, (23) pancreatic BxPC-3, (24) pancreatic CAPAN-2, (25) pancreatic CFPAC-1, (26) pancreatic HPAF-II, (27) pancreatic MIA PaCa-2, (28) pancreatic Panc-1, (29) pancreatic SU.86.86, (30) prostate DU-145, (31) prostate PC-3, (32) renal ACHN, (33) renal Caki, (34) uterine epithelial RL95-2.

FIG. 2 shows the expression of Ki-67 in thirty-four human tumor xenograft sections. See FIG. 1 legend for xenograft tumor type and cell line number.

FIG. 3 provides the immunohistochemical digital staining images, from left to right, for CD320, TCN2 and Ki-67 expression in each of thirty-four human tumor xenograft sections. See FIG. 1 legend for xenograft tumor type and cell line number.

FIG. 4 shows the expression of TCIIR (protein antibody=CD320) and TCII (protein antibody=TCN2) in A) naturally occurring tumors; and B) corresponding adjacent non-malignant tissues in thirty dogs: (1) anal gland adenocarcinoma 11090371, (2) anal gland adenocarcinoma 11091836, (3) anal gland adenocarcinoma 11101247, (4) digital squamous cell carcinoma 11110453, (5) digital squamous cell carcinoma 111120632, (6) digital squamous cell carcinoma 11120413, (7) fibrosarcoma 11120641, (8) fibrosarcoma 11120720, (9) fibrosarcoma 11120721, (10) hemangiosarcoma 11070302, (11) hemangiosarcoma 11101549, (12) hemangiosarcoma 11101711, (13) lymphoma 11111603, (14) lymphoma 11120588, (15) lymphoma 11120684, (16) melanoma (cutaneous) 11070597, (17) melanoma (oral) 11061907, (18) melanoma (oral) 11070304, (19) osteosarcoma 11061788, (20) osteosarcoma 11061908, (21) osteosarcoma 11070395, (22) prostate carcinoma 11032215, (23) prostate carcinoma 11041574, (24) prostate carcinoma 11071912, (25) thyroid carcinoma 11110836, (26) thyroid carcinoma 11111199, (27) thyroid carcinoma 11111344, (28) transitional cell carcinoma 10100415, (29) transitional cell carcinoma 10060321, (30) transitional cell carcinoma 11120309.

FIG. 5 shows the expression of Ki-67 in A) naturally occurring tumors; and B) corresponding adjacent non-malignant tissues in thirty dogs. See FIG. 4 legend for tumor type and case number.

FIG. 6 provides the immunohistochemical staining images for CD320, TCN2, and Ki-67 expression in thirty naturally occurring canine tumors as well as in corresponding adjacent non-malignant tissues. Canine tumor sections, from left to right, include: CD320 malignant, CD320 benign, TCN2 malignant, TCN2 benign, Ki-67 malignant, Ki-67 benign shown for each sample. 10× (200 um) magnification for CD320 & TCN2 and 40× (50 um) magnification for Ki-67 was used. The average staining intensity of 3 random, representative fields was determined for CD320 & TCN2 using an ImageJ color deconvolution plugin. ImageJ and an immunoratio plugin was used to evaluate Ki-67 staining. See FIG. 4 legend for tumor type and case number

FIG. 7 shows the expression of TCIIR (protein antibody=CD320) and TCII (protein antibody=TCN2) in A) naturally occurring tumors; and B) corresponding adjacent non-malignant tissues in thirty-six cats: (1) biliary carcinoma 10021255, (2) biliary carcinoma 10101918, (3) biliary carcinoma 10121242, (4) dermal carcinoma 10071506, (5) dermal carcinoma 11030645, (6) dermal carcinoma 11111180, (7) fibrosarcoma-vaccine 10051260, (8) fibrosarcoma-vaccine 11120550, (9) fibrosarcoma-vaccine 9070268, (10) intestinal adenocarcinoma 11100237, (11) intestinal adenocarcinoma 11101952, (12) intestinal adenocarcinoma 11120080, (13) intestinal lymphoma 11101365, (14) intestinal lymphoma 10120094, (15) intestinal lymphoma 11120384, (16) intestinal mast cell tumor 11071015, (17) intestinal mast cell tumor 11080200, (18) intestinal mast cell tumor 11111192, (19) mammary adenocarcinoma 11051222, (20) mammary adenocarcinoma 11051232, (21) mammary adenocarcinoma 11071104, (22) nodal lymphoma 11111342, (23) nodal lymphoma 11111667, (24) nodal lymphoma 11112001, (25) oral squamous cell carcinoma 11110315, (26) oral squamous cell carcinoma 11120118, (27) oral squamous cell carcinoma 11120336, (28) sarcoma 11041581, (29) soft tissue sarcoma 11011745, (30) soft tissue sarcoma 11091746, (31) splenic mast cell tumor 11031700, (32) splenic mast cell tumor 11050011, (33) splenic mast cell tumor 11050097, (34) transitional cell carcinoma-bladder 10060321, (35) transitional cell carcinoma-bladder 11011306, (36) transitional cell carcinoma-bladder 11041886.

FIG. 8 shows the expression of Ki-67 in A) naturally occurring tumors; and B) corresponding adjacent non-malignant tissues in thirty-six cats. See FIG. 7 legend for tumor type and case number.

FIG. 9 provides the immunohistochemical staining images for CD320, TCN2 and Ki-67 expression in thirty-six naturally occurring feline tumors as well as in the corresponding adjacent non-malignant tissues. Feline tumor sections, from left to right, include: CD320 malignant, CD320 benign, TCN2 malignant, TCN2 benign, Ki-67 malignant, Ki-67 benign shown for each sample. 10× (200 um) magnification for CD320 & TCN2 and 40× (50 um) magnification for Ki-67 was used. The average staining intensity of 3 random, representative fields was determined for CD320 & TCN2 using an ImageJ color deconvolution plugin. ImageJ and an immunoratio plugin was used to evaluate Ki-67 staining. See FIG. 7 legend for tumor type and case number.

### DETAILED DESCRIPTION OF THE INVENTION

The present invention provides methods for diagnosing, treating, and monitoring the progression of cancer based on the expression levels of a receptor that takes up vitamin B12, preferably transcobalamin II (TCII), transcobalamin II receptor (TCIIR), megalin, cubilin, amnionless and/or an asialoglycoprotein receptor and/or a proliferation marker such as Ki-67. In one embodiment, the selected markers are at least two of TCII, TCIIR, and/or Ki-67.

According to the invention, the expression levels of transcobalamin II (TCII), transcobalamin II receptor (TCIIR), Ki-67, megalin, cubilin, amnionless and/or an asialoglycoprotein receptor can be determined, for example, by measuring the amount of protein or mRNA for each of these markers.

According to one embodiment, the amount of transcobalamin II (TCII), transcobalamin II receptor (TCIIR), Ki-67, megalin, cubilin, amnionless and/or an asialoglycoprotein receptor protein in a test sample (and preferably a reference sample) is determined using antibodies specific for transcobalamin II (TCII), transcobalamin II receptor (TCIIR), Ki-67, megalin, cubilin, amnionless and/or an asialoglycoprotein receptor protein. Various techniques for measuring the amount of proteins using antibodies specific for the proteins are known in the art and are within the scope of the invention. For example, the techniques for measuring the amount of proteins using antibodies include, but are not limited to, immunohistochemical staining, flow cytometry, antibody-based arrays, enzyme-linked immunosorbent assay (ELISA), radioimmuno-assay (RIA), and western blotting. One or more of these techniques can be employed to determine the amount of marker proteins in a test sample and a reference sample according to the invention. Preferably, the amount of marker proteins in a test sample and a reference sample are determined using immunohistochemical staining or flow cytometry.

For example, we have found a statistically significant difference in immunohistochemical staining intensities for transcobalamin II (TCII), transcobalamin II receptor (TCIIR) and Ki-67 in malignant versus benign tissues. A variety of factors, including location of TCII production, TCII half life, and timing of TCIIR internalization can affect the quantity of either the transcobalamin transport protein or receptor present at the time of sampling. It is believed that these types of physiological variables affect the expression of each marker in each individual tumor, and so the aggregate expression information for multiple markers is preferred in diagnosis, treatment and progression monitoring.

The level of protein expression can be quantitated. For example, with immunohistochemical staining, the intensity of the stain is indicative of the level of protein expression. Thus, the quantitative level of expression for each marker can then be evaluated individually or combined with the expression level of the other markers to provide a “composite score” that can be determined by one of ordinary skill in the art without undue experimentation. The expression levels can be evaluated using the measured quantity of the test sample alone or in view of the reference sample.

Thus, one embodiment of the invention encompasses methods for diagnosing and/or treating cancer and/or monitoring the progression of a cancer, comprising:

a) determining the level of expression of transcobalamin II (TCII), transcobalamin II receptor (TCIIR), Ki-67, megalin, cubilin, amnionless and/or an asialoglycoprotein receptor in a test sample from a subject and optionally in a reference sample;

b) scoring the level of transcobalamin II (TCII), transcobalamin II receptor (TCIIR), Ki-67, megalin, cubilin, amnionless and/or an asialoglycoprotein receptor in each sample in a quantitative manner; and

c) diagnosing, treating or monitoring the subject based on an individual and/or composite score compared to a predetermined threshold value. The predetermined threshold value is preferably derived from clinical trials. For example, for a particular type of cancer, a value can be determined from clinical trials whereby a patient having an individual or composite score exceeding the threshold value has a high probability that a particular drug or family of drugs would be successful in treating that cancer.

The level of protein expression can also be “graded” or “scored” based on characteristic staining patterns using a scale from none/low to high, such as the four-category HercepTest™ (0, 1+, 2+, 3+), the Gleason system for degree of differentiation of prostate cancer cells, the Bloom-Richardson system or the Fuhrman system. Preferably, the highest score will be designated “positive” and the lowest score “negative” for each marker: transcobalamin II (TCII), transcobalamin II receptor (TCIIR), Ki-67, megalin, cubilin, amnionless and/or an asialoglycoprotein receptor. Whether intermediate scores are deemed positive or negative would depend, for example, on the scale used and the type of cancer involved. Determining whether a score is positive or negative for a given cancer can be determined by one of ordinary skill in the art without undue experimentation, preferably following clinical trials.

According to another embodiment, the expression levels of marker proteins are determined by measuring the amount of mRNA for each of these markers in a test sample and, preferably, a reference sample. In an embodiment, levels of marker protein mRNA in the test sample and the reference sample may be determined by extracting total RNA from the test sample and the reference sample and hybridizing the extracted RNA to labeled nucleic acid probes complementary to mRNA markers using any state of the art techniques. Techniques for detecting mRNAs using labeled nucleic acid probes include, but are not limited to, microarray analysis, northern blot analysis, nuclease protection assays, and fluorescence in situ hybridization. Alternatively, the levels of mRNA markers can be detected using reverse transcription-polymerase chain reaction (RT-PCR) with or without labeled nucleic acid probes. Additionally, newer techniques that do not rely on complementary target-probe hybridization, such as Next Generation Sequencing (NGS), may be used to determine levels of transcobalamin II (TCII), transcobalamin II receptor (TCIIR), Ki-67, megalin, cubilin, amnionless and/or an asialoglycoprotein receptor transcripts.

In another embodiment, the expression levels of marker proteins are determined by imaging of radiolabeled vitamin B12 analogs. Such methods are known to those of skill in the art.

Preferred embodiments of the invention begin with obtaining a test sample and a reference sample. The test sample and the reference sample could be a tissue sample, a hair sample, a serum sample, a blood sample, or any other body fluid sample. According to one aspect of the invention, the test sample and the reference sample may be obtained from the same subject. For example, the test sample may be obtained from the cancerous tissue of the subject and the reference sample may be obtained from the non-cancerous tissue of the same subject. According to another aspect, the test sample and the reference sample may be obtained from different subjects of the same species. For example, the test sample may be obtained from the cancerous tissue of the subject undergoing diagnosis and/or treatment and the reference sample may be obtained from a different subject of the same species who is healthy and not suffering from cancer. For example, the test sample may be obtained from a human subject suspected of having cancer and the reference sample may be obtained from another human subject.

The methods provided by the invention may be used for diagnosing, treating and monitoring various types of cancer, including but not limited to, tumors of the skin, soft tissues, bone, muscle, blood, brain, thyroid, adrenal gland, heart, lungs, esophagus, stomach, gall bladder, intestines, colon, rectum, spleen, liver, pancreas, kidneys, bladder, prostate, testicles, breasts, ovaries, uterus, cervix and lymph nodes. According to one aspect of the invention, once the subject is diagnosed as having cancer based on elevated expression levels of transcobalamin II (TCII), transcobalamin II receptor (TCIIR), Ki-67, megalin, cubilin, amnionless and/or an asialoglycoprotein receptor, the patient may be treated by administering a chemotherapeutic agent alone or in combination with one or more therapeutic agents. The chemotherapeutic agents that may be used include, but are not limited to, anthracyclines, alkylating agents, alkyl sulfonates, vinca alkaloids, nitrogen mustards, nitrosourea, antibiotics including cytotoxics, antimetabolites, folic acid analogs, vitamin B12 analogs, nucleotide analogs, precursor analogs, platinum-containing agents, cytoskeletal disruptors (taxanes), epothilones, histone deacetylase inhibitors, inhibitors of topoisomerase II, kinase inhibitors, angiogenesis inhibitors, proteosome inhibitors, cytostatic drugs such as etoposide, enzymes such as L-asparaginase, and monoclonal antibodies. Preferably, the chemotherapeutic agent is a vitamin B12-based anti-cancer agent. According to one embodiment, the chemotherapeutic agent is nitrosylcobalamin (NO-Cbl). The one or more therapeutic agents as indicated above include, but are not limited to, radiation therapy agents, angiogenesis inhibitors, hormones and monoclonal antibodies.

In one embodiment of the invention, one can identify a cancer patient who is a good candidate for treatment based on a metabolic pathway, comprising:

a) determining the level of transcobalamin II (TCII), transcobalamin II receptor (TCIIR), Ki-67, megalin, cubilin, amnionless and/or an asialoglycoprotein receptor in a test sample;

b) determining whether the test sample contains an overexpression of transcobalamin II (TCII), transcobalamin II receptor (TCIIR), Ki-67, megalin, cubilin, amnionless and/or an asialoglycoprotein receptor; and

c) identifying the patient as a good candidate for treatment based on a metabolic pathway if transcobalamin II (TCII), transcobalamin II receptor (TCIIR), Ki-67, megalin, cubilin, amnionless and/or an asialoglycoprotein receptor are overexpressed.

Examples of metabolic-based therapeutic agents include DNA-damaging agents, folate antagonists, analogs, homologs and derivatives of metabolic-based anti-cancer drugs, and the like. Thus, embodiments of the invention are useful in developing targeted, “personalized” diagnosis, treatment, monitoring and prognosis for patients.

The present invention is illustrated by the following examples, which are set forth to illustrate certain embodiments of the present invention and are not to be construed as limiting.

### EXAMPLES

### Example 1

### Immunohistochemical Quantification of TCII, TCIIR, and Ki-67 Expression in Human Tumor Xenografts

Commercial tissue microarray slides were purchased from the Tissue Acquisition and Cellular/Molecular Analysis Shared Service at the University of Arizona Cancer Center (Tucson, Ariz.). Expression of TCII, TCIIR, and Ki-67 were measured in thirty-four human tumor xenografts. Tumor cell lines evaluated included: (1) breast carcinoma MCF7/S, (2) breast carcinoma MDA-MB-231, (3) breast carcinoma ZR-75-1, (4) Burkitt's lymphoma Ramos, (5) cervical C-33A, (6) cervical Ca Ski, (7) cervical HeLa, (8) colon HCT-116, (9) colon HT-29, (10) epidermoid carcinoma A431NS, (11) Ewing's sarcoma CHP-100, (12) glioblastoma multiforme SF-767, (13) leukemia MDS/SP1, (14) lymphoma Granta-4, (15) lymphoma Raji, (16) melanoma A375, (17) non small cell lung cancer (NSCLC) H1975, (18) NSCLC MV-522, (19) NSCLC NCI: H460, (20) ovarian 2780AD, (21) ovarian carcinoma NIH-OVCAR-3, (22) pancreatic AsPC-1, (23) pancreatic BxPC-3, (24) pancreatic CAPAN-2, (25) pancreatic CFPAC-1, (26) pancreatic HPAF-II, (27) pancreatic MIA PaCa-2, (28) pancreatic Panc-1, (29) pancreatic SU.86.86, (30) prostate DU-145, (31) prostate PC-3, (32) renal ACHN, (33) renal Caki, and (34) uterine epithelial RL95-2.

Tumor xenografts were developed by injecting 10×10⁶ cancer cells into the flanks of severe combined immunodeficient mice. When the tumor volume of each xenograft approximated 500 mm³, mice were sacrificed by carbon dioxide asphyxiation and tumors were harvested. Tissues were fixed for 24 hours in 10% neutral buffered formalin, transferred to 70% histologic grade ethanol, processed overnight in a Tissue-Tek Tissue Processor, and embedded into paraffin blocks. A slide from each individual xenograft block was cut and stained with hematoxylin and eosin and reviewed by a board certified pathologist, who marked optimal areas for coring. A Tissue Microarray (TMA) block was made by removing a 2 mm core from each individual paraffin block and re-embedding multiple donor cores into a recipient TMA paraffin block. Three micron thick sections were cut from the donor TMA block and placed on positively charged glass slides. Slides were dried overnight at room temperature, followed by drying on a hot plate at 55-60° C. for 30 minutes.

For TCII and TCIIR analysis, slides were deparaffinized by processing in 2 changes of xylene for 5 minutes each, 2 changes of 100% histologic grade alcohol for 1 minute each, and 1 change of 95% histologic grade alcohol for 30 seconds and then rinsed with water. Endogenous peroxidase activity of the sections were quenched by incubation in 3% hydrogen peroxide for 15 minutes at room temperature. Following rinsing, slides were placed in citrate buffer of pH 6.0 and heat-induced epitope retrieval was accomplished using a Dako Pascal pressure chamber at 120° C. for 30 seconds. Once cooled, slides were rinsed with water, placed in wash buffer and loaded onto a Dako Autostainer Plus. In order to reduce nonspecific background staining, slides were blocked by sequential incubation at room temperature with avidin and biotin for 25 minutes each, followed by 10 minutes of incubation at room temperature with a protein block. Slides were then incubated for 1 hour at room temperature with the following primary antibodies: rabbit polyclonal antibody to TCII (TCN2; 1:50 dilution; Proteintech, Chicago, Ill.; catalog no. 12157-1-AP) or rabbit polyclonal antibody to TCIIR (CD320; 1:50 dilution; Proteintech, Chicago, Ill.; catalog no. 10343-1-AP). After rinsing with wash buffer, slides were incubated at room temperature with a biotinylated secondary antibody for 20 minutes, followed by an enzyme-streptavidin-horse radish peroxidase (HRP) conjugate for 20 minutes. Slides were then rinsed twice with wash buffer. Immunostaining visualization was achieved by immersion of slides in diaminobenzidine (DAB) for 5 minutes, followed by rinsing with wash buffer and counterstaining with Mayer's hematoxylin for 30 seconds. Slides were dehydrated and cleared by immersing in 95% histologic grade alcohol for 1 minute, 2 changes of 100% histologic grade alcohol for 1 minute each and 2 changes of xylene for 1 minute each. Slides were then mounted with a synthetic mounting medium. To ensure the specificity of immunostaining, a negative control TMA slide was prepared by omitting the primary antibody.

TCII and TCIIR-stained slides were examined by light microscopy (10×) with an Olympus microscope and an Olympus 12.5 megapixel, 12-bit digital color camera with Peltier cooling and using version 3.0 of DP Manager and Controller software. An ultra-high resolution mode (4080×3072) was utilized, and light settings were standardized for all imaging sessions. TCII and TCIIR positive cells exhibited DAB-positive (brown) staining; negative cells stained with the hematoxylin counterstain only. Stained slides were reviewed by a board certified pathologist. A representative area of solid tumor devoid of connective tissue and ischemic necrosis and with even distribution of cells was selected and digitally photographed for each tumor tissue sample. Digital images were obtained at 10× magnification (200 micron scale bar). JPEG format was used to capture all digital images. For each image, the color deconvolution method was used to isolate TCII and TCIIR-positive DAB-stained cells from TCII and TCIIR negative hematoxylin-stained cells. DAB and hematoxylin were digitally separated using ImageJ software (WS Rasband, National Institutes of Health, Bethesda, Md., URL http://rsb.info.nih.gov/ij/; version 1.46c) and an Image J plugin for color deconvolution, which calculated the contribution of DAB and hematoxylin based on specific red-green-blue (RGB) absorption. Following deconvolution, scale was set to the 200 micron scale bar on each image. The deconvoluted image was subjected to histogram analysis, with the lower threshold set at 10, and the upper threshold set at 100. For each image, three fields of consistent staining were selected and measured using 200×200 pixel boxes. A value was assigned to each field using ImageJ software, and the average value of all three fields was used to assign a staining value (arbitrary units) to each image.

For Ki-67 analysis, slides were deparaffinized in xylene and then rehydrated through 100%, 95% and 70% ethanol to 0.1M phosphate buffered saline (PBS). Prior to rehydration with 70% ethanol, slides were treated with 0.3% hydrogen peroxide in methanol for 30 minutes. Slides were placed into a citrate buffer antigen-retrieval solution of pH 6.0 and heat-induced epitope retrieval was accomplished using a steamer at 95° C. for 30 minutes. Slides were cooled for 20 minutes, rinsed in deionized water and placed in PBS. Slides were blocked with 10% normal horse serum in PBS with 0.02% Tween 20 for 20 minutes. One slide was incubated for 1 hour with a mouse monoclonal primary antibody to Ki-67 (Clone MIB-1; 1:40 dilution; Dako North America, Carpinteria, Calif.; catalog no. M7240); a second slide was not exposed to the primary antibody in order to serve as a negative control. Slides were rinsed three times with PBS-Tween 20 and incubated with a horse anti-mouse secondary antibody for 10 minutes. Slides were rinsed three times with PBS-Tween 20 and incubated with streptavidin-HRP for 10 minutes. Slides were rinsed three times with PBS-Tween 20, developed with Nova Red, counterstained for 15 seconds in Mayers hematoxylin and air dried.

Stained slides were examined microscopically as described above. Ki-67 positive cells exhibited Nova Red-positive (red-brown) staining. For quantification of Ki-67 expression, images were taken at 40× (50 micron scale bar) using the microscope, camera and resolution mode described above. Slide labels were omitted in order to minimize extraneous background staining. Images were analyzed using the ImageJ and ImmunoRatio plugin optimized for Ki-67 nuclear staining and utilizing the color deconvolution method (Tuominen V J et al., “ImmunoRatio: a publicly available web application for quantitative image analysis of estrogen receptor (ER), progesterone receptor (PR), and Ki-67, “Breast Cancer Res 2010, 12(4): p. R56). Background correction was performed using an image from an empty slide background (blankfield image) to address image color balance and uneven illumination. The number of Ki-67-positive Nova Red-stained cells over the total number of cells was calculated and used to assign a staining value (%) to each image.

The Spearman rank correlation coefficient was used as a non-parametric measure of the statistical dependence between TCII, TCII-R and Ki-67 expression in all xenograft tumors. Level of significance was set at p<0.05. Correlation coefficient values were calculated using an online computer software program (Wessa, P; 2012; Free Statistics Software, Office for Research Development and Education, version 1.1.23-r7).

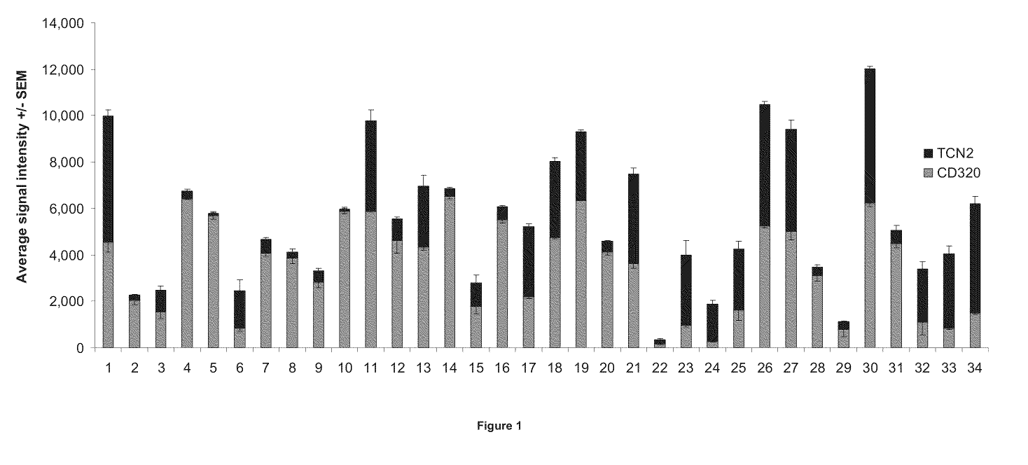

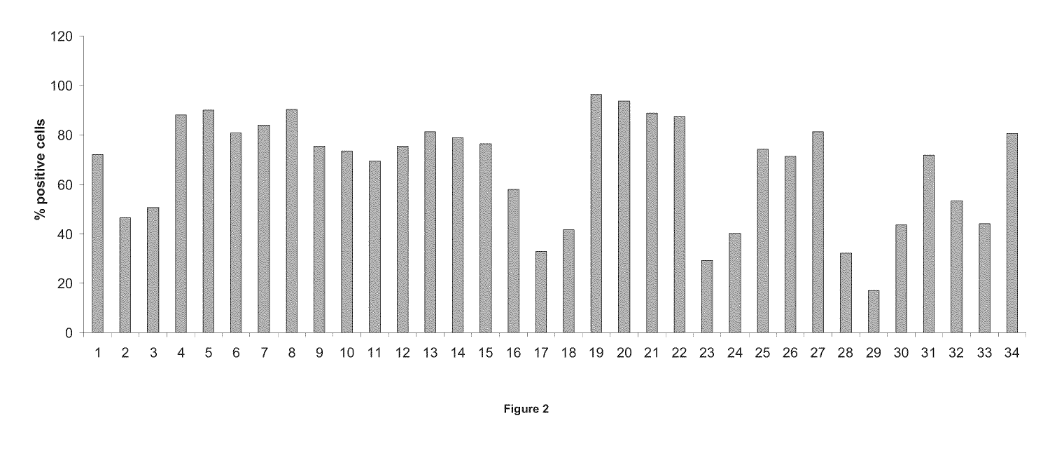

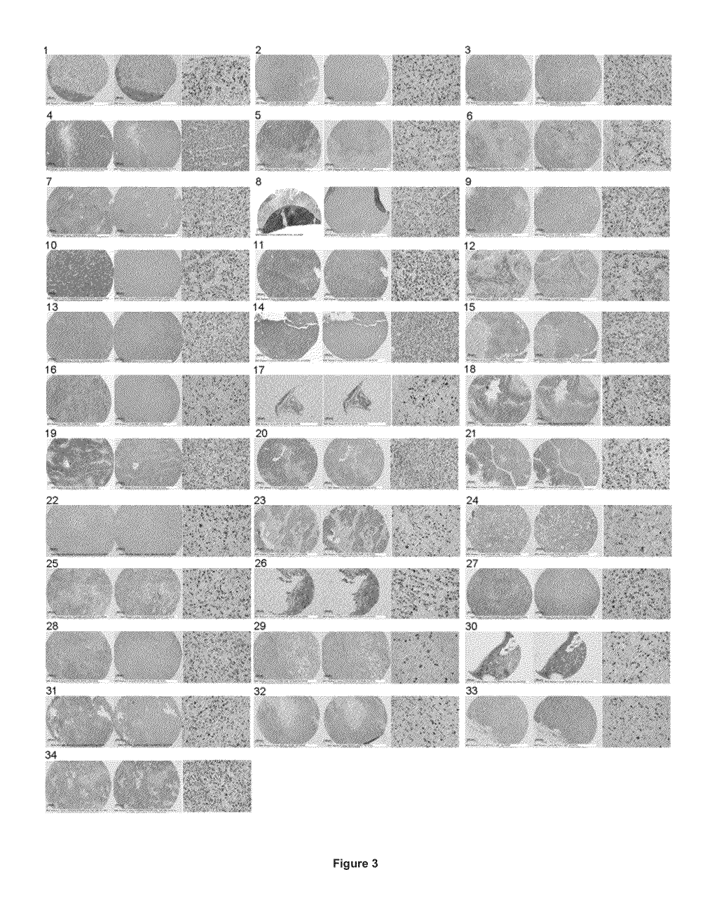

All tumor xenografts stained positively for TCII, TCIIR, and Ki-67 (FIGS. 1, 2, 3). Average/median staining values of xenograft tumor tissues were 1985/1310 (TCII), 3487/3962 (TCIIR) and 67%/74% (Ki-67). The range of staining values was 98-5793 (TCII), 149-6413 (TCIIR) and 17-96% (Ki-67). Immunohistochemical staining values for TCII, TCIIR and Ki-67 varied both between and within xenograft tumor types.

### Example 2

### Immunohistochemical Quantification of TCII, TCIIR, and Ki-67 Expression in Naturally Occurring Canine Tumors and in Corresponding Adjacent Non-Malignant Tissues

Expression levels of TCII, TCIIR, and Ki-67 were measured in ten types of spontaneously occurring canine tumor tissues as well as in corresponding immediately adjacent normal, non-malignant tissues. Tissue samples were obtained from the stored paraffin blocks of VDx Veterinary Pathology and Research Services Diagnostic Laboratory (Davis, Calif.). Three cases each of ten different canine tumor types (n=30) were selected based on previously established histopathological diagnosis. The ten tumor types evaluated included: (1) transitional cell carcinoma-bladder; (2) appendicular osteosarcoma; (3) melanoma; (4) splenic hemangiosarcoma; (5) anal gland adenocarcinoma; (6) lymphoma; (7) prostatic carcinoma; (8) fibrosarcoma; (9) digital squamous cell carcinoma; and (10) thyroid carcinoma.

Formalin-fixed, paraffin-embedded tissues were sectioned at 3 microns and mounted on positively charged glass slides; five slides were cut for each tumor. Slides were dried overnight at room temperature followed by drying on a hot plate at 55-60° C. for 30 minutes. One slide from each tumor was stained with hematoxylin and eosin and examined by a board certified pathologist to confirm the histopathological diagnosis and to verify that cut location included representative tumor tissue as well as adjacent normal, non-malignant tissue.

For TCII and TCIIR analysis, slides were deparaffinized by processing in 2 changes of xylene for 5 minutes each, 2 changes of 100% histologic grade alcohol for 1 minute each, and 1 change of 95% histologic grade alcohol for 30 seconds, and then rinsed with water. Endogenous peroxidase activity was quenched by incubation of the slides in 3% hydrogen peroxide for 15 minutes at room temperature. Following rinsing, slides were placed in citrate buffer of pH 6.0 and heat-induced epitope retrieval was accomplished using a Dako Pascal pressure chamber at 120° C. for 30 seconds. Once cooled, slides were rinsed with water, placed in wash buffer and loaded onto a Dako Autostainer Plus. Slides were incubated for 1 hour at room temperature with one of the following primary antibodies: rabbit polyclonal antibody to TCII (TCN2; 1:50 dilution; Proteintech, Chicago, Ill.; catalog no. 12157-1-AP) or with rabbit polyclonal antibody to TCIIR (CD320; 1:50 dilution; Proteintech, Chicago Ill.; catalog no. 10343-1-AP). After rinsing twice with wash buffer, slides were incubated for 1 hour with a rabbit-on-canine horseradish peroxidase (HRP) polymer secondary antibody. Slides were rinsed twice with wash buffer. Immunostaining visualization was achieved by immersion of slides in diaminobenzidine (DAB) for 5 minutes, followed by rinsing with wash buffer and counterstaining with Mayer's hematoxylin for 30 seconds. Slides were dehydrated and cleared by immersing in 95% histologic grade alcohol for 1 minute, 2 changes of 100% histologic grade alcohol for 1 minute each and 2 changes of xylene for 1 minute each. Slides were then mounted with a synthetic mounting medium. To ensure the specificity of immunostaining, negative control slides were prepared by omitting the primary antibody.

TCII and TCIIR-stained slides were microscopically examined and digitally photographed as described in Example 1, except that a representative, uniformly-stained area of adjacent non-malignant tissue was also photographed for each slide. Computer-assisted quantification of TCII and TCIIR expression in both tumor tissues and in corresponding adjacent non-malignant tissues was performed as described in Example 1.

For Ki-67 analysis, slide preparation, staining, digital photographing and computer-assisted quantification of Ki-67 expression were performed as described in Example 1 for tumor tissues as well as for corresponding adjacent non-malignant tissues.

Statistical analysis was performed as described in Example 1.

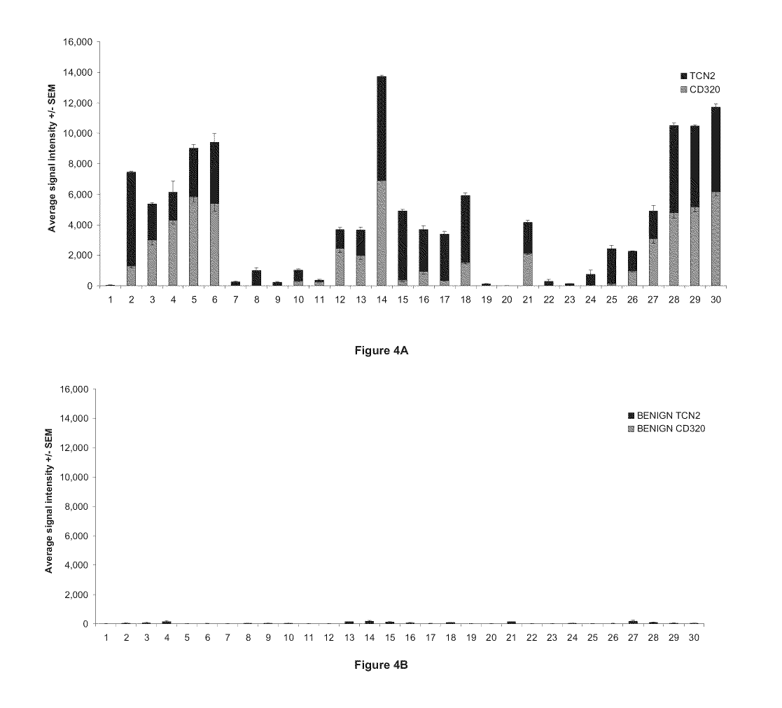

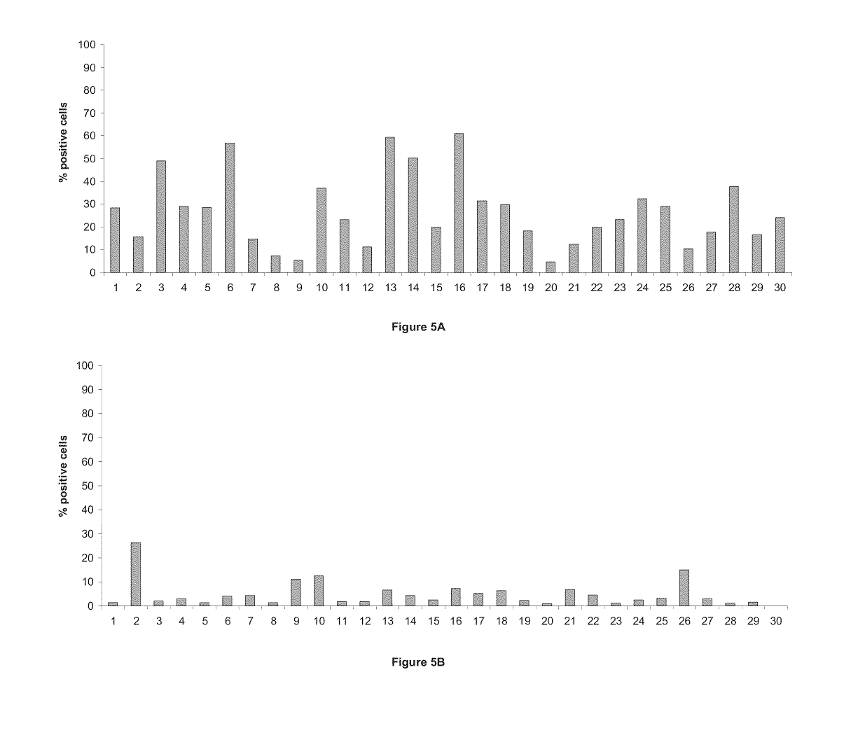

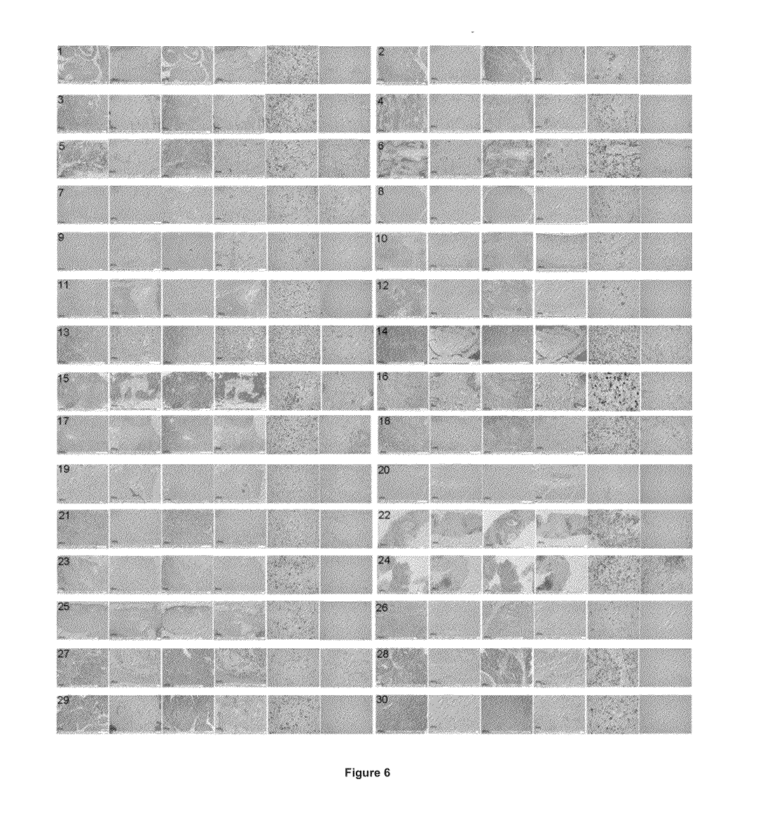

All canine tumors stained positively for TCII, TCIIR and Ki-67 (FIGS. 4A, 5A, 6), with the exception of one osteosarcoma case which did not express staining for TCII. There was no significant correlation between TCII, TCIIR or Ki-67 expression and breed, age, weight or sex. Immunohistochemical staining values for TCII, TCIIR and Ki-67 were relatively consistent within some tumor tissue types, and were more varied within others. Average/median staining values of canine tumor tissues were 2317/1824 (TCII), 1913/955 (TCIIR) and 27/23% (Ki-67); range of staining values was 0-6817 (TCII), 3-6897 (TCIIR) and 4-61% (Ki-67).

Canine tumors with the highest degree of TCII and TCIIR staining included transitional cell carcinoma of the bladder, digital squamous cell carcinoma and lymphoma. Tumors with a moderate degree of TCII and TCIIR staining included anal gland adenocarcinoma, thyroid carcinoma and melanoma. Tumors with the lowest TCII and TCIIR staining intensity were hemangiosarcoma, osteosarcoma, prostatic carcinoma and fibrosarcoma. In osteosarcoma cases, staining intensity was influenced by the amount of bone present in the sample (i.e. tumors with more osseous tissue in the sample exhibited less staining). The most proliferative canine tumors, as determined by the Ki-67 proliferative index, were lymphoma and melanoma. The least proliferative canine tumors included thyroid carcinoma, osteosarcoma and fibrosarcoma.

Adjacent benign tissues expressed significantly less staining for TCII, TCII-R and Ki-67 (FIGS. 4B, 5B, 6). Average/median staining values of adjacent benign tissues were 42/12 (TCII), 5/0.5 (TCIIR) and 5/3% (Ki-67); range of staining values was 0-151 (TCII), 0-47 (TCIIR) and 0-26% (Ki-67). For every case, there was a statistically significant difference between TCII, TCIIR and Ki-67 expression in tumor tissues compared to that in corresponding adjacent normal tissues (TCII: p<0.0001; TCIIR: p<0.1×10⁻⁵; Ki-67: p<0.1×10⁻⁷).

There was a statistically significant correlation between TCII and TCIIR expression in all tumor tissues (Spearman rank correlation coefficient value: 0.78; p<0.05). There was a modest correlation between TCII and Ki-67 expression in all tumor tissues (Spearman rank correlation coefficient value: 0.40, p<0.05) and between TCIIR and Ki-67 expression in all tumor tissues (Spearman rank correlation coefficient value: 0.41, p<0.05).

### Example 3

### Immunohistochemical Quantification of TCII, TCIIR, and Ki-67 Expression in Naturally Occurring Feline Tumors and in Corresponding Adjacent Non-Malignant Tissues

The expression levels of TCII, TCIIR, and Ki-67 were measured in twelve types of spontaneously occurring feline tumor tissues as well as in corresponding immediately adjacent normal, non-malignant tissues. Tissue samples were obtained from the stored paraffin blocks of VDx Veterinary Pathology and Research Services Diagnostic Laboratory (Davis, Calif.). Three cases each of twelve different feline tumor types (n=36) were selected based on previously established histopathological diagnosis. The twelve tumor types included: (1) biliary carcinoma; (2) dermal carcinoma; (3) vaccine-associated fibrosarcoma; (4) intestinal adenocarcinoma; (5) intestinal lymphoma; (6) intestinal mast cell tumor, (7) mammary adenocarcinoma; (8) nodal lymphoma; (9) oral squamous cell carcinoma; (10) soft tissue sarcoma; (11) splenic mast cell tumor; and (12) transitional cell carcinoma of the bladder.

For TCII and TCIIR analysis, slides were prepared and stained as described in Example 2, except that a 1:25 dilution was used for incubation with both TCN2 and CD320 antibodies. Digital photography of stained slides and computer-assisted quantification of TCII and TCIIR expression was performed as described in Example 2.

For Ki-67 analysis, slide preparation, staining, digital photography and computer-assisted quantification of Ki-67 expression were performed as described in Example 2.

Statistical analysis was performed as described in Example 2.

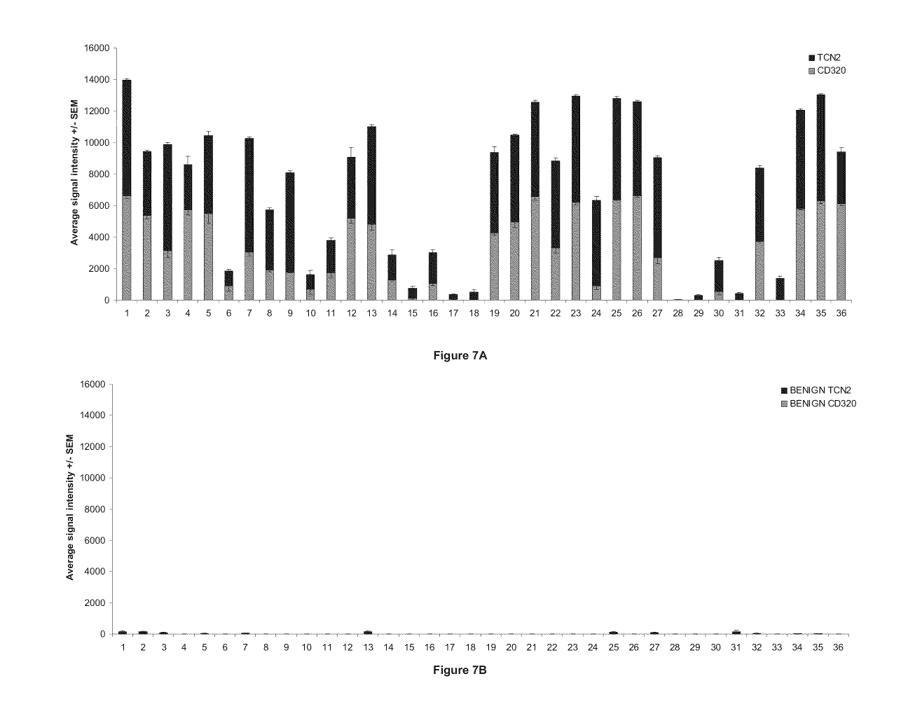

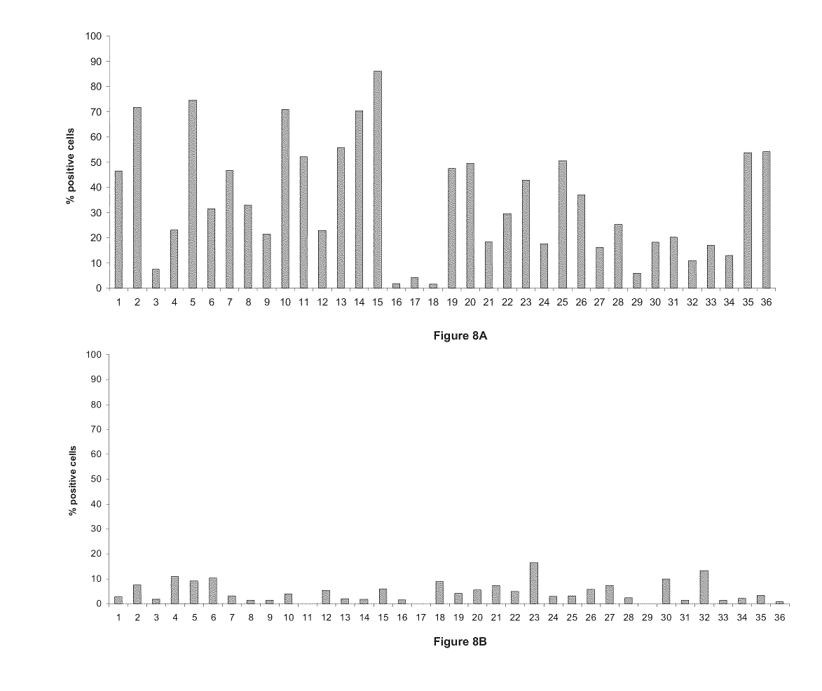

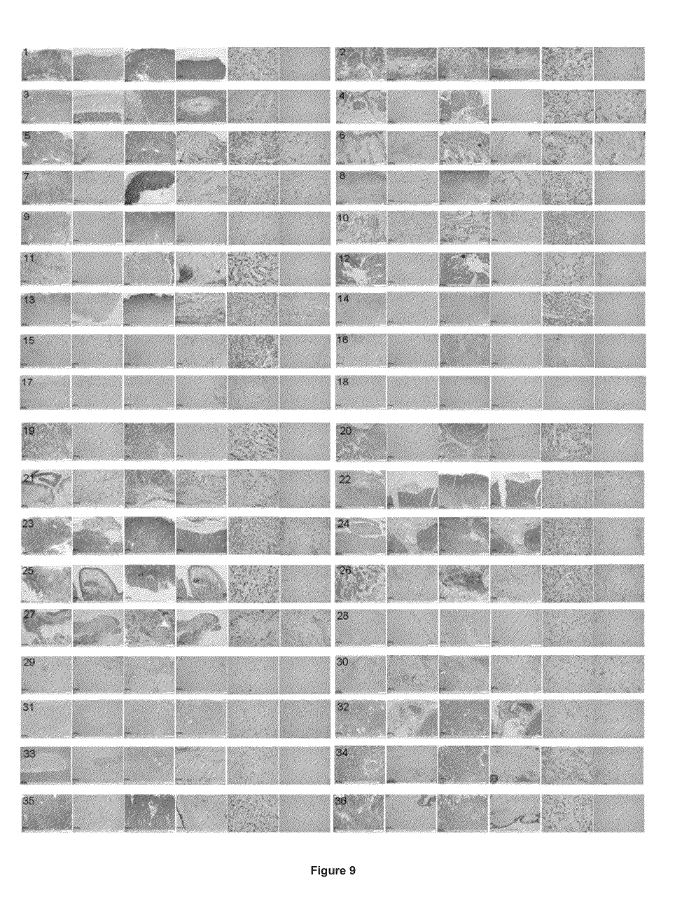

All feline tumors stained positively for TCII, TCII-R and Ki-67 (FIGS. 7A, 8A, 9), with the exception of one intestinal mast cell tumor case which did not express staining for TCIIR. There was no significant correlation between TCII, TCIIR or Ki-67 expression and breed, age or sex. Immunohistochemical staining values for TCII, TCIIR and Ki-67 were relatively consistent within some tumor tissue types, and were more varied within others. Average/median staining values of feline tumor tissues were 3899/4352 (TCII), 3149/3092 (TCIIR) and 35/31% (Ki-67); range of staining values was 36-7346 (TCII), 0-6622 (TCIIR) and 2-86% (Ki-67).

Feline tumors with the highest degree of TCII and TCIIR staining included transitional cell carcinoma of the bladder, mammary adenocarcinoma, oral squamous cell carcinoma and biliary carcinoma. Tumors with a moderate degree of TCII and TCIIR staining included nodal lymphoma, intestinal lymphoma, intestinal adenocarcinoma, dermal carcinoma and vaccine-associated fibrosarcoma. Tumors with the lowest TCII and TCIIR staining intensity were splenic mast cell tumor, intestinal mast cell tumor and soft tissue sarcoma. The most proliferative feline tumors, as determined by the Ki-67 proliferative index, were intestinal lymphoma, intestinal adenocarcinoma, dermal carcinoma, biliary carcinoma and transitional cell carcinoma of the bladder. The least proliferative feline tumors included splenic mast cell tumor, intestinal mast cell tumor and soft tissue sarcoma.

Adjacent benign tissues expressed significantly less staining for TCII, TCIIR and Ki-67 (FIGS. 7B, 8B, 9). Average/median staining values of adjacent benign tissues were 30/1 (TCII), 0.7/0 (TCIIR) and 5/3% (Ki-67); range of staining values was 0-166 (TCII), 0-9 (TCIIR) and 0-17% (Ki-67). For every case, there was a statistically significant difference between TCII, TCIIR and Ki-67 expression in tumor tissues compared to that in corresponding adjacent normal tissues (TCII: p<0.1×10⁻¹²; TCIIR: p<0.1×10⁻⁹; Ki-67: p<0.1×10⁻⁹).

There was a statistically significant correlation between TCII and TCIIR expression in all tumor tissues (Spearman rank correlation coefficient value: 0.76; p<0.05). There was a modest correlation between TCIIR and Ki-67 expression in all feline tumor tissues (Spearman rank correlation coefficient value: 0.36, p<0.05) but not between TCII and Ki-67 expression (Spearman rank correlation coefficient value: 0.15, p>0.05).

1. A method for diagnosing cancer in a subject, comprising:
a) for each of transcobalamin II (TCII), transcobalamin II receptor (TCIIR), Ki-67, megalin, cubilin, amnionless and/or an asialoglycoprotein receptor, determining the level of expression in a test sample from the subject and in a reference sample;
b) for each of transcobalamin II (TCII), transcobalamin II receptor (TCIIR), Ki-67, megalin, cubilin, amnionless and/or an asialoglycoprotein receptor, determining whether the level of expression is statistically significantly different for the test sample compared to the reference sample; and
c) diagnosing the subject as having cancer if the level of expression of transcobalamin II (TCII), transcobalamin II receptor (TCIIR), Ki-67, megalin, cubilin, amnionless, an asialoglycoprotein receptor, or a combination thereof, is statistically significantly different than the respective reference sample.

2. A method for treating cancer in a subject, comprising:
a) determining the level of expression of transcobalamin II (TCII), transcobalamin II receptor (TCIIR), Ki-67, megalin, cubilin, amnionless and/or an asialoglycoprotein receptor in a test sample from the subject and in a reference sample;
b) grading the level of each of transcobalamin II (TCII), transcobalamin II receptor (TCIIR), Ki-67, megalin, cubilin, amnionless and/or an asialoglycoprotein receptor as positive or negative and determining whether the level of expression is statistically significantly different for the test sample compared to the reference sample; and
c) administering a chemotherapeutic agent alone or in combination with one or more therapeutic agents to the subject if the expression of transcobalamin II (TCII), transcobalamin II receptor (TCIIR), Ki-67, megalin, cubilin, amnionless, an asialoglycoprotein receptor, or a combination thereof, is positive or is statistically significantly different than the respective reference sample.

3. A method for monitoring the progression of cancer in a subject, comprising:
a) determining the level of expression of transcobalamin II (TCII), transcobalamin II receptor (TCIIR), Ki-67, megalin, cubilin, amnionless and/or an asialoglycoprotein receptor in a test sample obtained from the subject at a first time point;
b) determining the level of expression of transcobalamin II (TCII), transcobalamin II receptor (TCIIR), Ki-67, megalin, cubilin, amnionless and/or an asialoglycoprotein receptor in a test sample obtained from the subject at a second time point, wherein the second time point is later than the first time point; and
c) evaluating the progression of cancer based on the change in expression level between the first and the second time points.

4. The method according to claim 3, wherein the subject is found to have i) a positive prognosis if the second expression level is statistically significantly lower than the first expression level, ii) a neutral prognosis if the second expression level is not statistically significantly different from the first expression level, or iii) a negative prognosis if the second expression level is statistically significantly higher than the first expression level.

5. The method according to claim 1, wherein the step of determining the level of expression of transcobalamin II (TCII), transcobalamin II receptor (TCIIR), Ki-67, megalin, cubilin, amnionless and/or an asialoglycoprotein receptor comprises determining the amount of transcobalamin II (TCII), transcobalamin II receptor (TCIIR), Ki-67, megalin, cubilin, amnionless and/or an asialoglycoprotein receptor proteins in the test sample and/or in the reference sample.

6. The method according to claim 5, wherein the amount of transcobalamin II (TCII), transcobalamin II receptor (TCIIR), Ki-67, megalin, cubilin, amnionless and/or an asialoglycoprotein receptor proteins is determined using antibodies specific for transcobalamin II (TCII), transcobalamin II receptor (TCIIR), Ki-67, megalin, cubilin, amnionless and/or an asialoglycoprotein receptor proteins.

7. The method according to claim 5, wherein the step of determining the level of transcobalamin II (TCII), transcobalamin II receptor (TCIIR), Ki-67, megalin, cubilin, amnionless and/or an asialoglycoprotein receptor expression employs immunohistochemical staining.

8. The method according to claim 5, wherein the step of determining the level of transcobalamin II (TCII), transcobalamin II receptor (TCIIR), Ki-67, megalin, cubilin, amnionless and/or an asialoglycoprotein receptor expression employs flow cytometry, antibody-based arrays, enzyme-linked immunosorbent assay (ELISA), radioimmuno-assay (RIA), western blotting, northern blot analysis, microarray analysis, nuclease protection assays, reverse transcription-polymerase chain reaction (RT-PCR) with or without labeled nucleic acid probes, or Next Generation Sequencing (NGS).

9. The method according to claim 5, wherein the step of determining the level of transcobalamin II (TCII), transcobalamin II receptor (TCIIR), Ki-67, megalin, cubilin, amnionless and/or an asialoglycoprotein receptor employs fluorescent in situ hybridization of mRNA.

10. The method according to claim 5, wherein the step of determining the level of transcobalamin II (TCII), transcobalamin II receptor (TCIIR), Ki-67, megalin, cubilin, amnionless and/or an asialoglycoprotein receptor employs radiolabeled vitamin B12 imaging.

11. The method according to claim 1, wherein the test sample and the reference sample is selected from a tissue sample, a hair sample, a serum sample, a blood sample, or any other body fluid sample.

12. The method according to claim 1, wherein the value of the reference sample is derived from clinical trials.

13. The method according to claim 1, wherein the reference sample is obtained from said subject or a different subject of the same species.

14. The method according to claim 13, wherein the reference sample is obtained from a non-cancerous tissue of the same subject.

15. The method according to claim 2, wherein the chemotherapeutic agent is a cobalamin drug conjugate.

16. The method according to claim 2, wherein the chemotherapeutic agent is selected from the group consisting of: anthracyclines, alkylating agents, alkyl sulfonates, vinca alkaloids, nitrogen mustards, nitrosourea, antibiotics such as cytotoxic antibiotics, antimetabolites, folic acid analogs, vitamin B12 analogs such as nitrosylcobalamin, nucleotide analogs and precursor analogs, platinum-containing agents, cytoskeletal disruptors (taxanes), epothilones, histone deacetylase inhibitors, inhibitors of topoisomerase II, kinase inhibitors, angiogenesis inhibitors, proteosome inhibitors, cytostatic drugs such as etoposide, enzymes such as L-asparaginase, and monoclonal antibodies.

17. The method according to claim 16, wherein the chemotherapeutic agent is nitrosylcobalamin.
